(19) United States
(12) Patent Application Publication
(10) Pub. No.: US 2022/0186174 A1
(43) Pub. Date: Jun. 16, 2022

(54) ACOUSTIC EDGE EFFECT

(71) Applicant: FloDesign Sonics, Inc., West Springfield, MA (US)

(72) Inventors: Bart Lipkens, Hampden, MA (US); Benjamin Ross-Johnsrud, Northampton, MA (US); Kedar C. Chitale, Little Rock, AR (US); Krishna N. Kumar, Wilbraham, MA (US); Jack Saloio, Ludlow, MA (US)

(21) Appl. No.: 17/610,572

(22) PCT Filed: May 15, 2020
(86) PCT No.: PCT/US2020/033181

§ 371 Date: Nov. 11, 2021

Related U.S. Application Data
(60) Provisional application No. 62/848,462, filed on May 15, 2019.

(51) Int. Cl. C12M 1/00; C12N 13/00; B01D 21/28; B01D 21/30

(52) U.S. Cl. CPC C12M 47/02; B01D 21/30; B01D 21/283; C12N 13/00

(57) ABSTRACT

An acoustic wave with an acoustic field with a large number of multi-directional gradients can provide an edge effect that be used to form an interface region relative to the acoustic wave. The interface region can block material with certain characteristics related to the nature of the interface region. Other material that is less influenced by the acoustic properties of the interface region can pass through the acoustic wave. This technique permits separation of materials using the edge effect and interface region.

### BACKGROUND

Separation of biomaterial has been applied in a variety of contexts. For example, separation techniques for separating proteins from other biomaterials are used in a number of analytical processes.

Acoustophoresis is a technique for separating particles and/or secondary fluids from a primary or host fluid using acoustics waves, such as ultrasonic waves. Acoustic waves can exert forces on particles in a fluid when there is a differential in density and/or compressibility, known as the acoustic contrast factor. The pressure profile in an acoustic wave contains areas of local minimum pressure amplitudes at wave nodes and local maxima at wave anti-nodes. Depending on their density and compressibility, the particles can be driven to and trapped at the nodes or anti-nodes of the acoustic wave. Generally, the higher the frequency of the standing wave, the smaller the particles that can be trapped.

### BRIEF DESCRIPTION

This disclosure describes technologies relating to methods, systems, and apparatus for acoustic separation of materials. The materials being separated may be biomaterials. An acoustic wave is generated in a fluid, forming pressure differentials at different locales. In a flowing fluid, a pressure rise may be generated on an upstream interface region where the acoustic wave interacts with the flowing fluid. The acoustic wave generates an acoustic radiation force acting on material suspended in the fluid. The pressure rise and acoustic radiation force forms a tunable barrier or filter at the interface region. This phenomenon is referred to herein as an interface effect or edge effect, interchangeably. The interface effect the barrier or filter characteristics of the interface effect is located at the upstream of the fluid flow. Characteristics of the acoustic wave may be modified to change or control characteristics of the interface region and interface effect. For example, a frequency of the acoustic wave may be controlled to cause materials with a particular acoustic contrast factor to be blocked by or to be passed through the acoustic wave. As another example, characteristics of the acoustic wave can be modified to block or pass materials of a given size range, while blocking or passing materials of another, different size range.

In some examples, the material blocked or retained by the interface effect is particles, which may be designed to work with the interface effect to achieve a certain result. As used herein, the term “particles” refers to any type of material that is differentiated from the fluid in which the material is suspended. Particles may be used as support structures for other compounds or biomaterial. Particles may be beads that may include a rigid component, such as glass, a polymer or paramagnetic material, or may include a pliable component, such as liquids or gases, including oils or lipids. A functionalized material may be applied to the support structures that has an affinity for one or more materials to be separated. The support structures may be mixed in a fluid that contains the materials. The fluid mixture may be presented to an interface region formed with an acoustic wave, such as by being flowed through a fluid chamber. The interface region formed by the acoustic wave can differentiate, for example, block or pass, the support structures, in relation to other material in the fluid.

In some examples, material adhered to the support structures with the functionalized material remains in the column, while other free material in the fluid may pass through the acoustic wave to provide separation of materials. The support structures may be implemented to have a certain acoustic contrast factor based on their density, compressibility, size or other characteristics that permits the support structures to react more strongly to the acoustic standing wave than other materials in the fluid mixture.

An acoustic transducer can be used to generate the acoustic wave, which can generate pressure forces in one or multiple directions. In multiple directions, the acoustic standing wave forces can be of the same order of magnitude. For example, forces in the direction of wave propagation may be of the same order of magnitude as forces that are generated in a different direction. An interface region can be generated near a border or edge of the acoustic wave that contributes to preventing support structures from passing. Multiple transducers may be used, some for generating an acoustic wave in one or more modes, and/or others for generating an acoustic wave in another, different mode. For example, the acoustic wave can be a standing wave that can generate pressure forces in one dimension or direction, or in multiple dimensions or directions. The acoustic wave can be generated in a mode to form an interface region to prevent passage of certain materials while permitting passage of other materials. The acoustic wave can be generated in a mode to trap and cluster certain materials that build in size until the gravity or buoyancy forces on the clusters surpass the other forces on the clusters, such as fluidic or acoustic forces, so that the clusters drop or rise out of the acoustic wave.

The particles may include biomaterial, such as cells, and may include support structure/biomaterial complexes. An acoustic transducer that includes piezoelectric material for vibrating at ultrasonic frequencies may be used to produce the acoustic wave. The acoustic transducer may be operated in one or more modes to obtain a desired effect and/or result. For example, the acoustic transducer may be operated in a mode to preferentially trap or block particles with certain density, size, compressibility, and/or other characteristics. The trapped or blocked particles may be collected using the acoustic transducer operating in a collection mode, where the particles rise or settle out of the acoustic wave due to being clustered and enlarged in size to enhance buoyancy or gravity forces acting on the clustered particles such that the enhanced forces exceed the acoustic and/or fluid drag forces. The rising or settling of particles can be advantageously exploited to collect the separated particles and remove them from the fluid chamber. The mode of trapping particles for separation by rising or settling out of the acoustic wave may be accompanied by a mode of blocking or passing the particles in a fluid path. The mode of preventing or permitting passage may be implemented with an acoustic wave with an interface region across the fluid path.

An example apparatus may include a fluid chamber configured to receive fluid containing functionalized material. The fluid chamber may be in the form of a column. An acoustic transducer is arranged in relation to the fluid chamber, for example, acoustically coupled to the fluid chamber, to provide an acoustic wave or signal into the fluid chamber when excited. Excitation of the transducer in certain modes can generate a multi-directional acoustic field in the fluid in the fluid chamber that includes a number of spatial locales where acoustic pressure amplitude is differentiated. For example, some spatial locales may possess a relatively high acoustic pressure amplitude compared to other spatial locales that obtain a relatively low acoustic pressure amplitude.

In some example modes, the particles may be driven to and retained at some of the spatial locales of the multi-directional acoustic field. In some example modes, the particles may be blocked from entering or crossing an acoustic field generated by the acoustic wave by the interface effect. In some example modes, the particles may be passed through the acoustic field generated by the acoustic wave due to fluid drag forces, buoyancy forces, and/or gravity forces. The fluid chamber may be configured for operation in different orientations. For example, where buoyancy or gravity forces are used in fluid chamber operation, the fluid chamber may be vertically oriented. The fluid chamber may be arranged at an angle to vertical. Such an angled arrangement may provide advantages for fluid dynamics management or deployment of the interface effect. Vertical flow which may be in an upward or downward direction. An acoustic transducer can be coupled to an end of the fluid chamber to permit the formation of a fluidized bed or expanded bed in the fluid chamber. In such an arrangement, a particle-fluid mixture can be flowed into the fluid chamber, and the acoustic wave produced by the acoustic transducer can prevent target particles from exiting the fluid chamber with the fluid flow, to thereby form an expanded or fluidized bed in the fluid chamber.

In some examples, an ultrasonic transducer is coupled to a flow path provided in a fluid chamber. The ultrasonic transducer is excited to generate an acoustic wave in the flow path. In filtration terms, fluid and material passing through and exiting the acoustic wave is referred to as the filtrate or permeate and material blocked or retained by the acoustic wave is referred to as the concentrate or retentate. The acoustic wave may be a standing or traveling wave, may be planar or multi-directional, or a combination of such waves. The acoustic transducer may be operated in a higher order mode to produce a multi-directional acoustic wave, or may be operated in “piston” mode to produce a uni-directional or linear acoustic wave. When operating in a higher order mode, a waveform is induced on a surface of the active element of the acoustic transducer, thereby launching acoustic waves in multiple directions. In piston mode, the surface of the active element moves in a uniform back and forth motion, thereby launching an acoustic wave in a single direction. In some examples, the ultrasonic transducer is configured to generate a multi-directional acoustic wave that has an acoustic radiation force with an axial force component and a lateral force component that are of the same order of magnitude.

In the case of an acoustic standing wave, a reflector of acoustic energy may be located opposite to the acoustic transducer. The reflector may be situated across a fluid chamber or flow path from the acoustic transducer. The reflector may be planar and reflect acoustic energy at a reflection angle that is the same as the incident angle. The reflector may be composed of a number of elements arranged at different angles or that extend away from the reflector different distances. Such a complex reflector may reflect acoustic energy at different reflection angles depending on the element of the reflector to which the incoming acoustic wave is incident. A complex reflector may be designed and implemented to obtain a specific response, for example to form a specifically shaped or positioned interface region, or to enhance or diminish the interface effect. In addition, or alternatively, multiple ultrasonic transducers may be used to generate acoustic waves in the fluid chamber or flow path. For example, an ultrasonic transducer may be located opposite to another ultrasonic transducer to contribute to generating an acoustic standing wave therebetween. Such a second ultrasonic transducer may be passive to reflect acoustic energy, or maybe active to contribute to generating and/or controlling an acoustic standing wave. In addition, or alternatively, multiple ultrasonic transducers may be positioned along the fluid chamber or flow path to permit generation of multiple acoustic waves at different locations.

Methods for forming an interface region and interface effect are also disclosed herein. For example, the interface region can be formed by operating an ultrasonic transducer in a collection mode to capture and retain particles in a fluid flow against fluid drag force. The collected particles can be used to form a pressure differential Acoustic transducer a method for using acoustic radiation force to build up a pressure rise upstream of the interface region

In an example implementation, separation devices are provided that are adapted to (i) receive a mixture containing a primary fluid and acoustically sensitive material; and (ii) employ an acoustic standing wave to block the acoustically sensitive material as the mixture is presented to the acoustic standing wave, thereby changing the concentration of material in the fluid. The concentration change produces a pressure rise on the upstream interface region of the acoustic standing wave to contribute to forming an interface region. Acoustic radiation force acting on the incoming suspended material further contributes to forming the interface region. The interface effect that forms the interface region acts as a barrier to the suspended material.

The location of the interface region can be modulated and controlled using various operating parameters. For example, the fluid dynamics of the mixture can be controlled by various column design techniques, controlling fluid velocity and/or controlling the characteristics of the fluid mixture. In addition, or alternatively, implementation of the acoustic wave can be controlled to influence various characteristics of the interface region. For example, a control can be implemented to cause the interface region to be formed upstream of the position of the ultrasonic transducer, or to be formed to overlap the position of the ultrasonic transducer. The frequency of the acoustic wave may be modified such that different contrast factor materials may be held back or allowed through the acoustic wave, or such that particles of one given size range are retained and particles of a second given range are allowed to flow through the acoustic wave. The acoustic waves that produce the interface effect to form the interface region may also be modulated so as to let selective materials through at different times or with different velocities during operation.

Example implementations discussed herein include separation devices that possess at least one inlet for receiving a mixture of fluid, target material and non-target material, an ultrasonic transducer that produces an ultrasonic acoustic wave and uses a pressure rise and an acoustic radiation force to generate an interface region to separate the target material from the mixture. The example separation devices include an outlet port for output of non-target material during separation, and for output of target material in a harvesting phase. The fluid mixture may comprise particles such as mammalian cells, bacteria, cell debris, fines, proteins, exosomes, vesicles, viruses, plant cells and insect cells.

In some example implementations, the separation device includes a recirculation path to permit additional dwell time for target material in the fluid chamber or flow path. The separation device can be operated in several modes, including an initial loading mode to load a mixture of fluid and particles, a sample loading mode to introduce biomaterial into the separation device, a recirculation mode to enhance capture of target material, and an eluting mode to harvest the captured target material.

The details of one or more implementations of the subject matter described in this specification are set forth in the accompanying drawings and the description below.

### BRIEF DESCRIPTION OF THE DRAWINGS

Drawings are presented for the purposes of illustrating embodiments disclosed herein and not for the purposes of limiting the same.

FIG. 1 is a diagram of a material separation device implementing an acoustic edge effect.

FIG. 2 is a diagram of a material separation device initiating an acoustic edge effect with a perfluorohexane suspension.

FIG. 3 is two side-by-side images of the implementation of an acoustic pressure profile, the left-hand side being an image of a physical implementation and the right-hand side being an image of a simulated implementation.

FIG. 4 is an image of simulated acoustic pressure gradients contributing to an acoustic edge effect.

FIG. 5 is a diagram of acoustic pressure and radiation forces for establishing an acoustic edge effect.

FIG. 6 is an analytical diagram of an acoustic interface.

FIG. 7 is a simulation model of acoustic wave lateral forces for a positive acoustic contrast particle.

FIG. 8 is a simulation model of acoustic wave lateral forces for a negative acoustic contrast particle.

FIG. 9 is a graph illustrating acoustic radiation force exerted at an acoustic interface region.

FIG. 10 is a force diagram for a body in an acoustic field.

FIG. 11 is a graph of interface force versus volume fraction for a perfluorohexane suspension.

FIG. 12 is a graph of interface force versus volume fraction for a polystyrene suspension.

FIG. 13 is a graph of breakthrough curves for different sized particles.

FIG. 14 is a graph of breakthrough curves for different particle concentrations.

FIG. 15 is a graph of breakthrough curves for different particle concentrations and different sized particles.

FIG. 16 is a graph of a breakthrough curve for Sepharose beads.

FIG. 17 is a partial front elevation view of an operating acoustic separation device showing an acoustic edge effect.

FIG. 18 is an isometric view of a faceted reflector.

FIGS. 19, 20, 21, and 22 are partial front elevation views of an operating acoustic separation device showing different acoustic interface region locations.

FIG. 23 is a diagram of an acoustic separation device implementing an acoustic edge effect for particles with a density greater than water.

FIG. 24 is a diagram of an acoustic separation device implementing an acoustic edge effect for particles with a density less than water.

FIG. 25 is a diagram of an acoustic separation device implementing an acoustic edge effect with no fluid flow.

FIG. 26 is a diagram of an acoustic separation device implementing an acoustic edge effect with fluid flowing.

FIG. 27 is a diagram of acoustic separation devices with varying geometries and fluid dynamics.

FIG. 28 is a diagram of an acoustic separation device illustrating vortices at an acoustic interface region.

FIG. 29 is a partial front elevation view of an acoustic separation device with multiple interface regions.

FIG. 30 is a flowchart of a process for initiating an acoustic edge effect.

FIG. 31 is a diagram of an acoustic separation device configured for affinity separation of cellular material.

FIG. 32 is a diagram of an acoustic separation device and process for affinity separation of cellular material with perfluorohexane droplets.

FIG. 33 is a graph of purity and recovery for TCR—cellular material.

### DETAILED DESCRIPTION

The present disclosure may be understood more readily by reference to the following detailed description of desired embodiments and the examples included therein. In the following specification and the claims which follow, reference will be made to a number of terms which shall be defined to have the following meanings.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

The term “comprising” is used herein as requiring the presence of the named component and allowing the presence of other components. The term “comprising” should be construed to include the term “consisting of”, which allows the presence of only the named component, along with any impurities that might result from the manufacture of the named component.

Numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values). The endpoints of the ranges and any values disclosed herein are not limited to the precise range or value; they are sufficiently imprecise to include values approximating these ranges and/or values.

The modifier “about” used in connection with a quantity is inclusive of the stated value and has the meaning dictated by the context. When used in the context of a range, the modifier “about” should also be considered as disclosing the range defined by the absolute values of the two endpoints. For example, the range of “from about 2 to about 10” also discloses the range “from 2 to 10.” The term “about” may refer to plus or minus 10% of the indicated number. For example, “about 10%” may indicate a range of 9% to 11%, and “about 1” may mean from 0.9-1.1.

It should be noted that many of the terms used herein are relative terms. For example, the terms “upper” and “lower” are relative to each other in location, i.e. an upper component is located at a higher elevation than a lower component in a given orientation, but these terms can change if the device is flipped. The terms “inlet” and “outlet” are relative to a fluid flowing through them with respect to a given structure, e.g. a fluid flows through the inlet into the structure and flows through the outlet out of the structure. The terms “upstream” and “downstream” are relative to the direction in which a fluid flows through various components, i.e. the flow fluids through an upstream component prior to flowing through the downstream component. It should be noted that in a loop, a first component can be described as being both upstream of and downstream of a second component.

The terms “horizontal” and “vertical” are used to indicate direction relative to an absolute reference, i.e. ground level. However, these terms should not be construed to require structures to be absolutely parallel or absolutely perpendicular to each other. For example, a first vertical structure and a second vertical structure are not necessarily parallel to each other. The terms “top” and “bottom” or “base” are used to refer to surfaces where the top is always higher than the bottom/base relative to an absolute reference, i.e. the surface of the earth. The terms “upwards” and “downwards” are also relative to an absolute reference; upwards is always against the gravity of the earth.

The present application refers to “the same order of magnitude.” Two numbers are of the same order of magnitude if the quotient of the larger number divided by the smaller number is a value of at least 1 and less than 10.

Briefly, the present disclosure relates to acoustic separation devices capable of generating an acoustic edge effect from one or more piezoelectric transducers. In some examples, the transducer is electrically excited into a multi-mode displacement pattern of vibration to generate a multi-directional acoustic wave. Alternatively, or in addition, the acoustic transducer may be excited in a piston mode to generate a planar or uni-directional acoustic wave. Combinations of acoustic waves may be generated, such as a combination of planar and multi-directional acoustic waves. The acoustic waves can be used to generate an interface effect or edge effect, these terms being used herein interchangeably. The interface effect causes the formation of an interface region with an acoustic force field that blocks material from passing through the acoustic wave and the accompanying acoustic field. The interface effect can be configured to target certain types of material, such as by blocking material that has characteristics of density, compressibility and/or size in specific ranges. The interface effect can be configured to pass certain types of material, such as by being configured to influence such material to a much smaller degree than other specific material to be blocked. The material to be passed experiences a much smaller degree of influence from the acoustic wave and acoustic field, so that other forces dominate, such as gravity, buoyancy or fluid drag forces.

The acoustic edge effect results in an acoustic radiation force that can overcome the combined effects of fluid drag and buoyancy or gravity at certain flow rates. As a result, the radiation force acts as a filter that prevents targeted particles (e.g., biological cells or acoustically responsive beads) from crossing through the standing wave. Several forces produced by the acoustic wave can have an impact on the acoustic edge effect and the generated interface region. For example, a multi-directional acoustic wave can produce lateral forces that act on material in a fluid flow presented to the acoustic wave. As an acoustic field generated by the acoustic wave impinges on material, a scattering effect of the acoustic field off the particles in the material produces a three-dimensional acoustic radiation force. The acoustic radiation force produces a net force effect on the particle. Nonzero net force may cause the particle to be moved in the acoustic field, and a close to zero net force may cause the particle to be trapped in the acoustic field. The acoustic radiation force is proportional to the particle volume (e.g., the cube of the radius) when the particle is small relative to the wavelength. The acoustic radiation force is proportional to frequency and the acoustic contrast factor of the material. The acoustic radiation force scales with acoustic energy (e.g., the square of the acoustic pressure amplitude). For harmonic excitation, the sinusoidal spatial variation of the force is what drives the particles to the stable positions within the acoustic waves, particularly acoustic standing waves. Particle trapping can occur when the acoustic radiation force exerted on the particles is greater than the combined effect of fluid drag force and buoyancy/gravitational force on the particle. In some modes of operation, the action of the lateral and axial acoustic forces generated by the acoustic wave on the trapped particles results in formation of tightly packed clusters through concentration, clustering, clumping, agglomeration and/or coalescence of particles that, when reaching a critical size, settle continuously through enhanced gravity for particles heavier than the host fluid or rise out through enhanced buoyancy for particles lighter than the host fluid. Additionally, secondary inter-particle forces, such as Bjerkness forces, aid in particle agglomeration. The clustering of particles can contribute to formation of the interface region and enhancing the acoustic edge effect.

In the devices of the present disclosure, during startup, the fluid ensonified by the acoustic standing wave is clarified by the process of trapping particles in clusters that grow in size until gravitational or buoyancy forces dominate acoustic and/or fluid drag forces. At such a point, the particle clusters are driven out of the acoustic wave. The acoustic standing wave is a three-dimensional acoustic field, which, in the case of excitation by a rectangular transducer, can be described as occupying a roughly rectangular prism or cuboid volume of fluid. Typically, two opposing faces are the transducer and reflector, an adjacent pair of opposing faces are the walls of the device, and the final opposing pair of faces, the upstream and downstream faces of the cuboid, though which the fluid flow is provided. The interface region is generally located more in an upstream region in relation to the acoustic standing wave field. This location is also referred to as an upstream interface region. The clarified fluid that passes through the acoustic wave is downstream of the interface region. Various combinations of operating parameters such as flow velocity, electrical power applied to the transducer, fluid concentration or configuration of the acoustic wave, to name just a few examples, can influence the location and characteristics of the interface region.

The transducer design can affect performance of the system. A typical transducer is a layered structure with the ceramic piezoelectric element bonded to a backing layer and a wear plate. Because the transducer is loaded with the high mechanical impedance presented by the standing wave, the traditional design guidelines for wear plates, e.g., half wavelength thickness for standing wave applications or quarter wavelength thickness for radiation applications, and manufacturing methods may not be appropriate. Rather, in some example embodiments of the present disclosure the transducers have no wear plate or backing, allowing the piezoelectric element to vibrate in one of its eigenmodes with a high Q-factor, or in a combination of several eigenmodes. The vibrating ceramic piezoelectric element/disk may be directly exposed to the fluid flowing through the separation device.

Removing the backing (e.g. making the piezoelectric element air backed) also permits the ceramic piezoelectric element to vibrate at higher order modes of vibration with little damping (e.g. higher order modal displacement). In a transducer having a piezoelectric element with a backing, the piezoelectric element vibrates with a more uniform displacement, like a piston. Removing the backing allows the piezoelectric element to vibrate in a non-uniform displacement mode.

The size, shape, and thickness of the piezoelectric material determines the transducer displacement at different frequencies of excitation. In some example implementations, the transducer is operated at frequencies near the thickness resonance frequency (half wavelength). A greater number of acoustic force gradients can be produced by a transducer operating in a higher order mode. Higher order modal displacements generate acoustic waves with strong gradients in the acoustic field in multiple directions, thereby creating acoustic radiation forces in multiple directions.

The lateral force of the acoustic radiation force generated by the transducer can be increased by driving the transducer in higher order mode shapes, as opposed to a form of vibration where the piezoelectric material (e.g., a piezoelectric crystal) effectively moves as a piston having a uniform displacement. The acoustic pressure is proportional to the driving voltage of the transducer. The electrical power is proportional to the square of the voltage. The transducer is typically a thin piezoelectric plate, with electric field in the z-axis and primary displacement in the z-axis. The transducer is typically coupled on one side by air (i.e., the air gap within the transducer) and on the other side by a fluid mixture. The types of waves generated in the plate are known as composite waves. A subset of composite waves in the piezoelectric plate is similar to leaky symmetric (also referred to as compressional or extensional) Lamb waves. The piezoelectric nature of the plate typically results in the excitation of symmetric Lamb waves. The waves are leaky because they radiate into the fluid layer, which result in the generation of the acoustic waves in the fluid layer. Lamb waves exist in thin plates of infinite extent with stress free conditions on its surfaces. Because the transducers of this embodiment are finite in nature, the actual modal displacements are more complicated.

The in-plane displacement (x-displacement) and out-of-plane displacement (y-displacement) across the thickness of the plate, the in-plane displacement being an even function across the thickness of the plate and the out-of-plane displacement being an odd function. Because of the finite size of the plate, the displacement components vary across the width and length of the plate. In general, a (m,n) mode is a displacement mode of the transducer in which there are m undulations in transducer displacement in the width direction and n undulations in the length direction. The maximum number of m and n is a function of the dimension of the piezoelectric material (e.g., a piezoelectric crystal) and the frequency of excitation. Additional three-dimensional modes exist that are not of the form (m,n).

The transducers are driven so that the piezoelectric element vibrates in higher order modes of the general formula (m, n), where m and n are independently 1 or greater. Higher order modes produce acoustic waves in a number of directions, resulting in a greater number of node and antinode locations, characterized by strong gradients in the acoustic field.

In some example implementations, the ultrasonic transducer(s) are driven by an electrical signal, which may be controlled based on voltage, current, phase angle, power, frequency or any other electrical signal characteristic. In particular, the driving signal for the transducer may be based on voltage, current, magnetism, electromagnetism, capacitive or any other type of signal to which the transducer is responsive. In embodiments, the signal driving the transducer can have a sinusoidal, square, sawtooth, pulsed, or triangle waveform; and have a frequency of 500 kHz to 10 MHz.

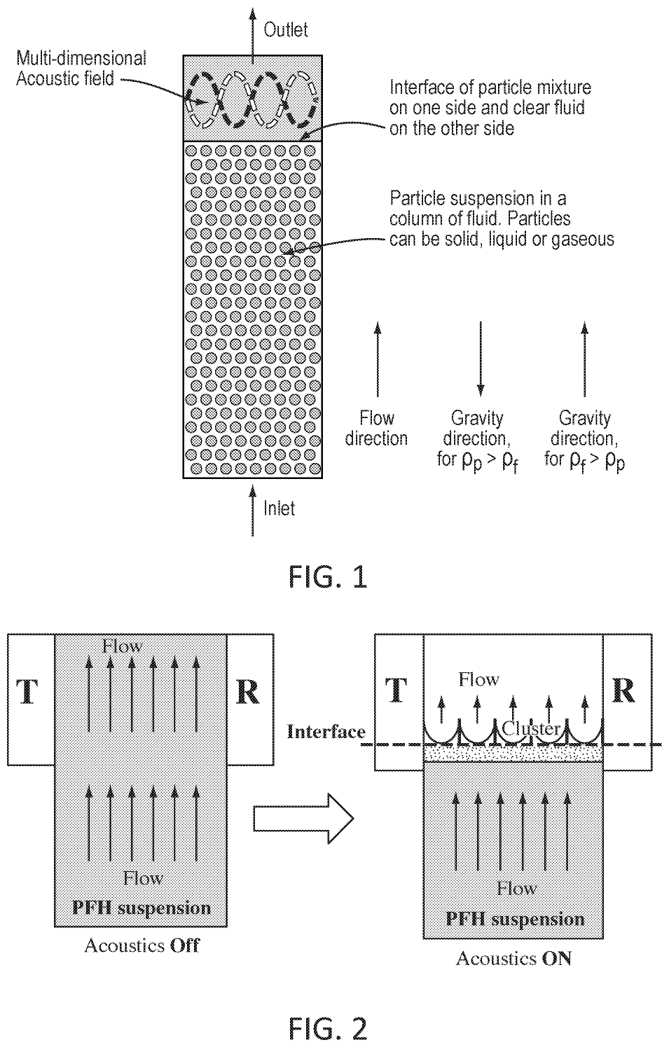

Referring to FIG. 1, a diagram of an acoustic separation device implementing an acoustic edge effect is illustrated. Acoustic edge effect: Acoustic radiation forces form an interface between mixture of particles and fluid on one side and clear fluid on the other side. The radiation forces in the flow direction arise from the lateral standing wave field component of the multidimensional field; diffracted waves by the transducer and scattered waves of a non-planar reflector crossing the interface lead to additional radiation forces on the particles. Acoustic radiation forces exert a downward force on the particles which is stronger than the fluid drag forces on the particles, potentially aided by the gravitational force, thereby maintaining a steady interface. Acoustic forces obstruct particles from moving into the acoustic field. Relatively low concentration of particles may be used to establish this effect (e.g., ˜1-2%). One or more transducers and one or more reflectors can be used. The acoustic field can be at an angle. With acoustics on, the flow is turned on, the acoustic edge holds the particles back and allows the clear fluid to pass through, an effect similar to filtration. The acoustic edge can be below the transducer or in the acoustic field as well.

Some of the parameters that influence formation and stability of the interface region include concentration of particles, acoustic pressure/power, flow rate, acoustic contrast factor of the material, the size of particles in the material, transducer frequency, and properties of the medium.

FIG. 2 illustrates operation of an acoustic separation device to form an interface region with a perfluorohexane (PFH) suspension. In the diagram on the left-hand side, the acoustic transducer T is not energized and fluid flows through the device unimpeded. In the diagram on the right-hand side, the acoustic transducer is energized to form an interface region with the acoustic edge effect. The interface region produces acoustic forces to which the PFH particles are highly responsive. The blocking of the PFH particles at the interface region contributes to increasing the acoustic edge effect to stabilize the interface region.

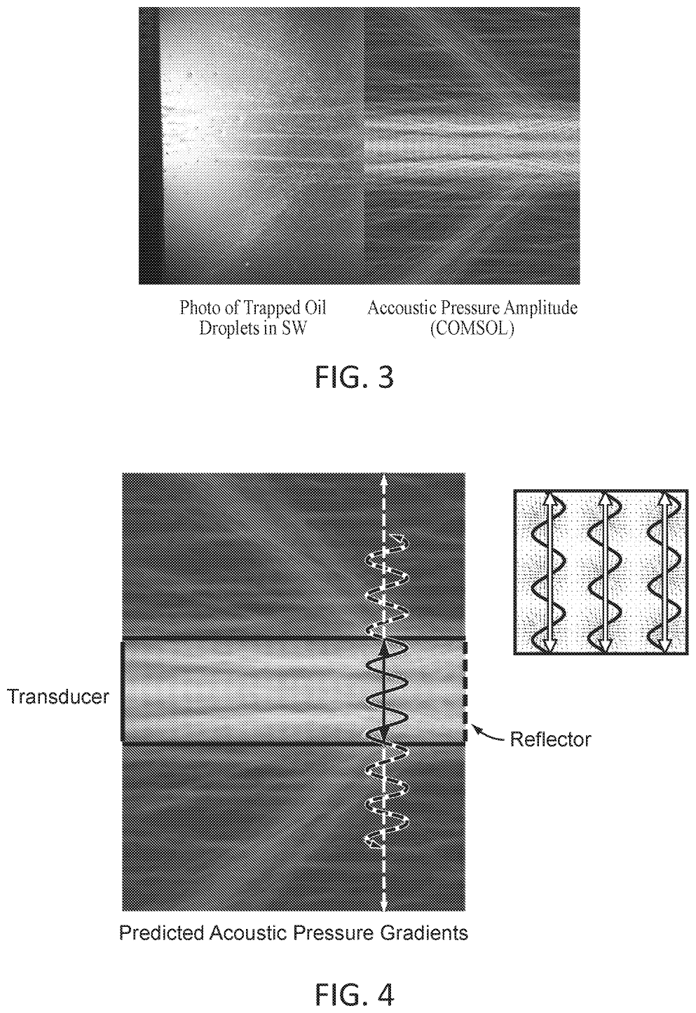

FIG. 3 illustrates the agreement of experimental and simulation data for production of the edge effect. The experimental and simulation data confirm that significant gradients are formed in the vertical direction, or laterally to the axial direction of the acoustic wave. The pressure in the acoustic resonator for the experiment is described by the following equation.

p_{w}(x, y, z)=ρ_{w}w²(ψ₋e^{-ik}^{_{z}}^{z}+ψ₊e^{ik}^{_{z}}^{z})cos(kₓx)cos(k_{y}y)

FIG. 4 illustrates a simulation to show predicted acoustic pressure gradients, including lateral radiation forces on particles to contribute to forming an interface region. Lateral pressure gradients and lateral wave strength are much larger in the standing wave. Axial standing wave forces can have an impact on and increase lateral wave strength. Lateral waves transmitted out of an acoustic standing wave decreased in strength. The interface region can therefore be formed with an edge at a standing wave lower edge due to lateral wave force reduction.

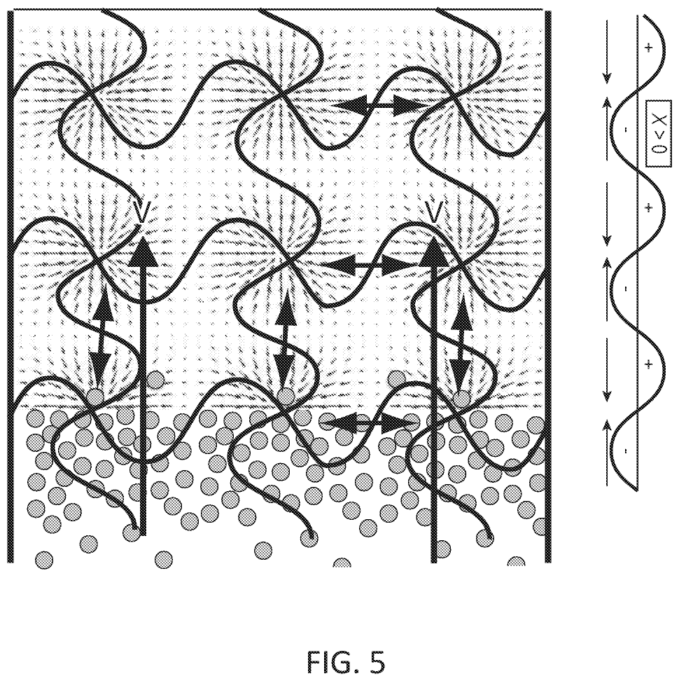

FIG. 5 illustrates acoustic forces generating gradients in the acoustic separation device to form the interface region. Gradients are weak below bottom clusters due to transducer mounting constraints. Moving lateral waves push the particle edge to the lower axial acoustic wave edge.

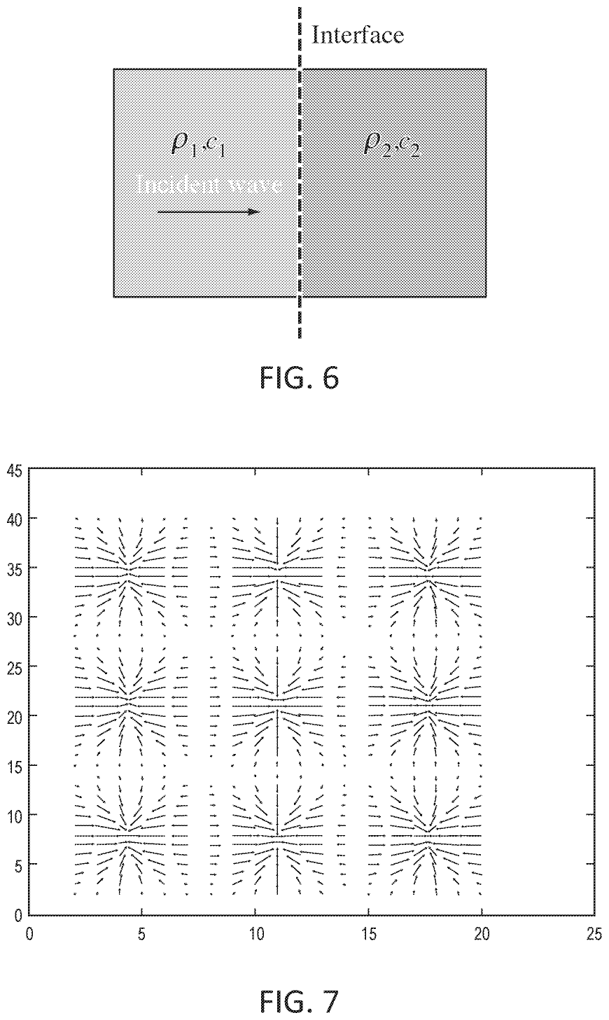

FIG. 6 shows a diagram for an analytical model of the acoustic edge effect. The force generated at the interface is determined with the following equation.

$${F}_{1 \to 2} = 2 \frac{{p}_{1}^{2}}{{ρ}_{1} {c}_{1}^{2}} [ \frac{{ρ}_{1}^{2} {c}_{1}^{2} + {ρ}_{2}^{2} {c}_{2}^{2} - 2 {ρ}_{1} {ρ}_{2} {c}_{1}^{2}}{{( {ρ}_{1} {c}_{1} + {ρ}_{2} {c}_{2} )}^{2}} ] A$$

ρ and c—depends on volume fraction(ϕ) of PFH at interface

This force contribution is related to the difference of properties between two fluids and is independent of the size of particles. Through experiments, particle size has been shown to have a significant effect on the edge effect. For certain mixtures (e.g., polystyrene particles-mixture) this force will be additive to the lateral radiation force, for other mixtures (e.g., PFH droplets) it will be subtractive, meaning this force is opposite to the lateral radiation force. Along with standing waves, there are traveling waves that cross the interface. This phenomenon produces a force on the interface, derived from the radiation pressure at the interface between the clear fluid and the mixture.

The pressure in the standing wave for a typical rectangular transducer is given by the following equations.

$$P = A cos ( {k}_{x} x ) cos ( {k}_{y} y ) cos ( {k}_{z} z ) {e}^{j w t}$$

$${k}_{x} = \frac{n π}{{d}_{x}} , {k}_{y} = \frac{m π}{{d}_{y}} , {k}_{z} = \frac{l π}{L}$$

In the above equations, dx=length in X direction, dy=length in Y direction, L=Path length of the standing wave, kₓ, k_{y}, k_{z} are wave numbers in those directions, n,m,l are the mode numbers taking values from 0, 1, 2, 3 e.g. For a 3×3 mode n=m=3. The lateral radiation forces obstruct the particles from moving into the acoustic field. The particles stay below the acoustic edge forming an interface as only clear fluid is allowed to pass through. The particles form a “self clearing channel” as the flow brings in more particles from the bottom, the ones at the top fall due to the action of gravity leading to mixing. The particles do not clog or stagnate at the acoustic edge and are continuously cleared out. The acoustics and flow can be periodically turned off to allow the particles to settle to “force” clearing of the acoustic edge. The transducer can be non-rectangular as well, e.g., circular, hexagonal, octagonal, fractal geometry, it can be any shape, and similar calculations can be done to determine radiation forces in all directions.

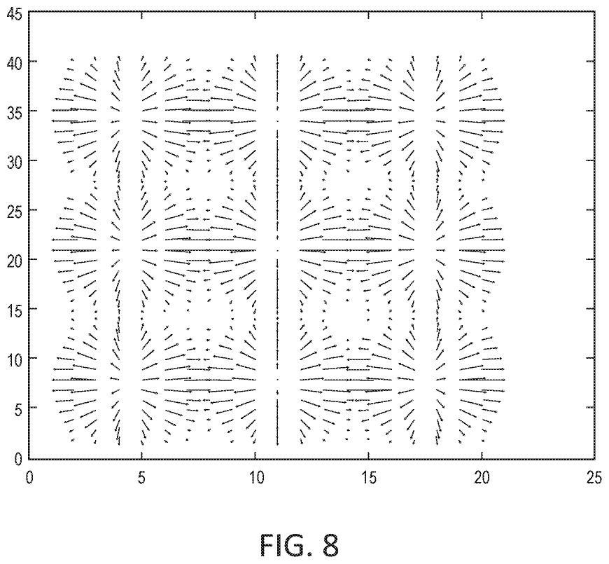

FIG. 7 shows a diagram of lateral force field components for a positive acoustic contrast particle. FIG. 8 shows a diagram of lateral force field components for a negative acoustic contrast particle. The locations where the particles find stable positions are thus different for positive contrast and negative contrast particles in the lateral planes.

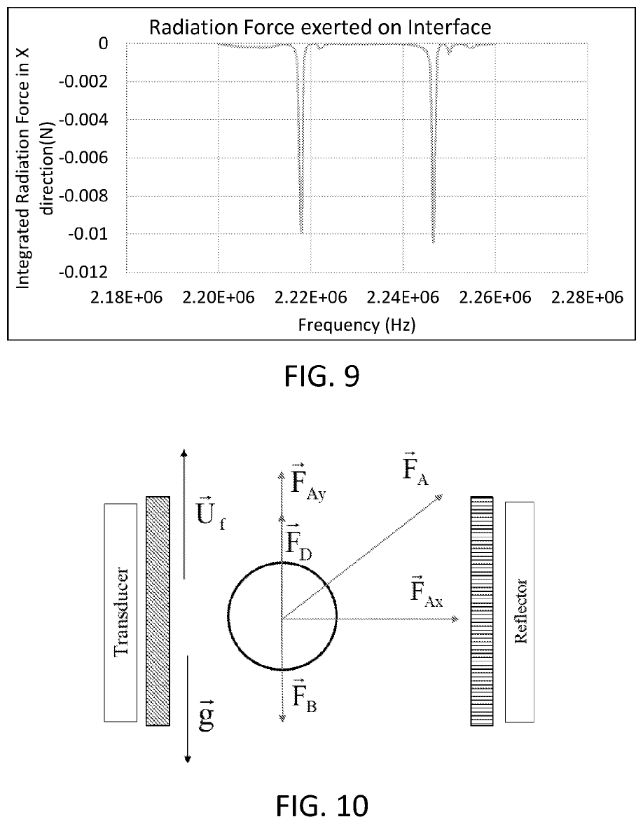

FIG. 9 is a graph of radiation force exerted on the particles at the interface region as a function of frequency. Force is calculated on the interface assuming a single particle approximation and integration along the interface. The force is negative in the x-direction, meaning it opposes the fluid drag forces, and that the particles are obstructed from entering the acoustic field, thereby confirming the existence of the interface effect. An example simulation implemented to determine force at the interface region a frequency of 2.16 MHz verifies the assumption of a pressure release boundary condition at the edge of the acoustic field.

FIG. 10 is a diagram of the forces on a particle in an acoustic field. The standing wave field produces acoustic radiation forces on particles in the axial as well as the lateral directions. The lateral radiation forces in the direction of the flow are responsible for the acoustic trapping of the particles in the acoustic field. The lateral radiation forces come from the vibration of the PZT/crystal/transducer in the lateral directions. Any combination of transducer, reflector assemblies that enhance lateral radiation forces can be suitable for the acoustic edge effect including the inclusion of scatterers in the field, non-planar reflectors, focused transducers and focused reflectors. The following equations describe the forces contributing to the acoustic edge effect.

$${\vec{F}}_{D} = 6 π {μ}_{f} {R}_{P} ( {\vec{U}}_{f} - {\vec{U}}_{P} ) \\ {\vec{F}}_{B} = \frac{4}{3} π {R}_{P}^{3} ( {ρ}_{p} - {ρ}_{f} ) \vec{g} \\ Gor ’ kov ’ s {Formulation}^{1}$$

$${\vec{F}}_{A} = - ∇ U , U = {V}_{0} [ \frac{〈 {p}^{2} ( x , y , t ) 〉}{2 {ρ}_{f} {c}_{f}^{2}} {f}_{1} - \frac{3 {ρ}_{f} 〈 {v}^{2} ( x , y , t ) 〉}{4} {f}_{2} ]$$

$${f}_{1} = 1 - \frac{1}{Λ {σ}^{2}} {f}_{2} = \frac{2 ( Λ - 1 )}{2 Λ + 1}$$

$$σ = \frac{{c}_{p}}{{c}_{f}} Λ = \frac{{ρ}_{p}}{{ρ}_{f}}$$

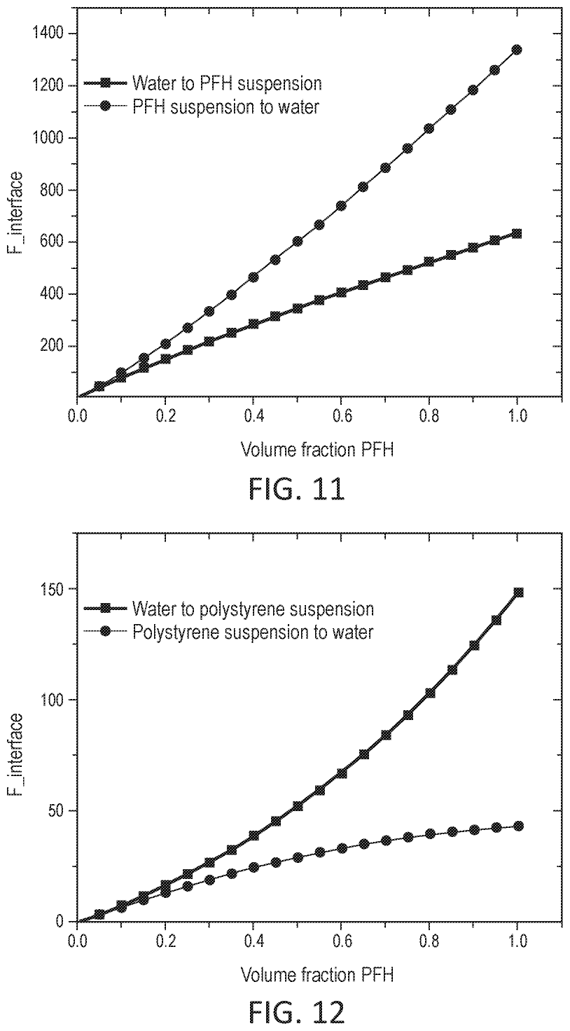

FIG. 11 is a graph illustrating the force at the interface for PFH beads suspended in water as the volume fraction of the PFH beads is changed. The PFH beads are denser than water, and have a negative acoustic contrast factor.

FIG. 12 is a graph illustrating the force at the interface for polystyrene beads suspended in water as a volume fraction of the polystyrene beads is changed. The polystyrene beads are denser than water and have a positive acoustic contrast factor.

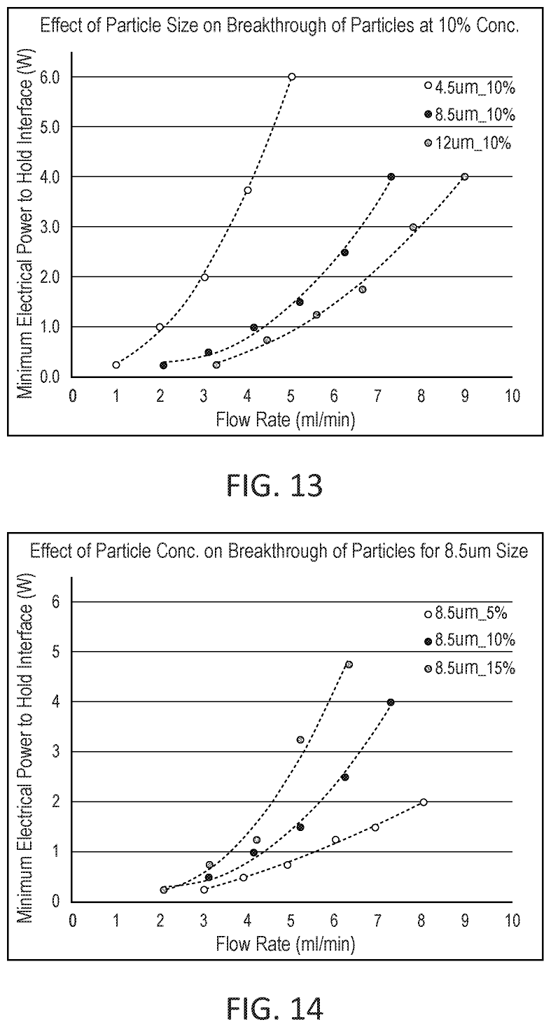

FIG. 13 is a graph showing breakthrough curves for different bead sizes at the same concentration. The acoustic interface region is stable at lower powers for the same flow rate as the particle size increases. This result follows as the acoustic force on a particle scales with particle size (R²).

FIG. 14 is a graph showing breakthrough curves for beads that are 8.5 μm in size, but at different concentrations. The acoustic interface region is stable at lower powers for the same flow rate as the droplet concentration is reduced. The stability results from an increase in velocity due to larger obstruction to the fluid flow by higher concentrations of particles. The flux rate of particles into the interface zone increases with increased particle concentration, implying a greater acoustic force field strength to maintain the acoustic edge effect.

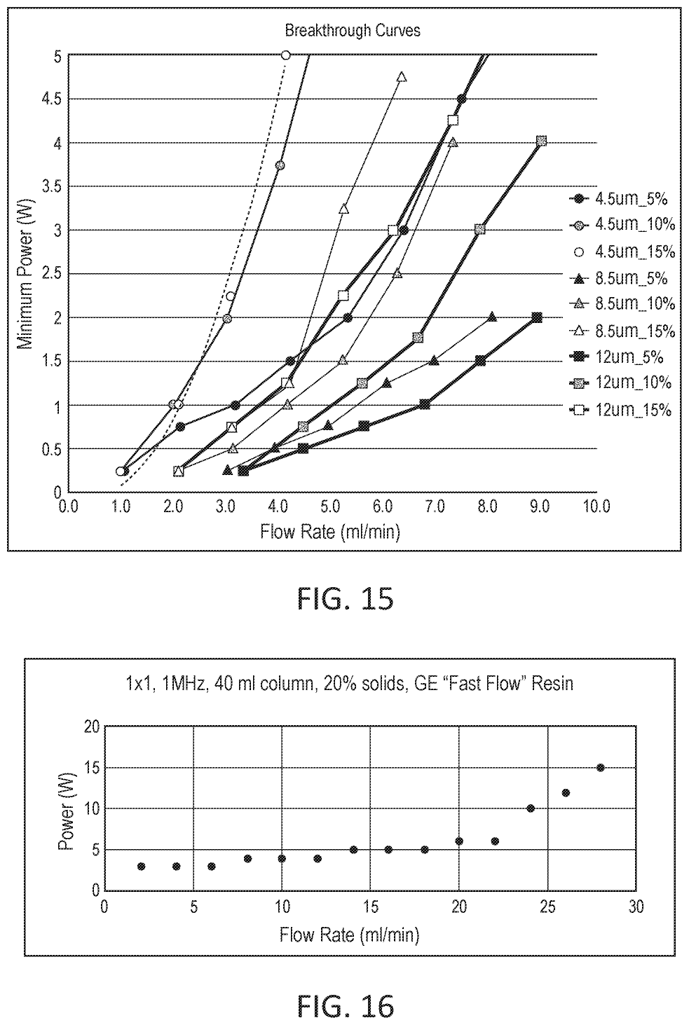

FIG. 15 is a graph showing the combination of breakthrough curves with varying bead sizes and varying concentrations. The combinations of lower concentrations and larger particle sizes imply less power used to set up and maintain the acoustic edge effect. The power to flow rate ratio, as illustrated in FIG. 15, can be used as a guide set operating parameters for the acoustic separation device based on bead size and concentration of beads used in the fluid mixture.

FIG. 16 is a graph showing a breakthrough curve for Sepharose beads. Sepharose beads are denser than water and have a positive acoustic contrast factor. Sepharose beads are bio-digestible, which may provide an advantage for cell and gene therapies where biomaterial collected using Sepharose beads is introduced into a patient as a therapeutic.

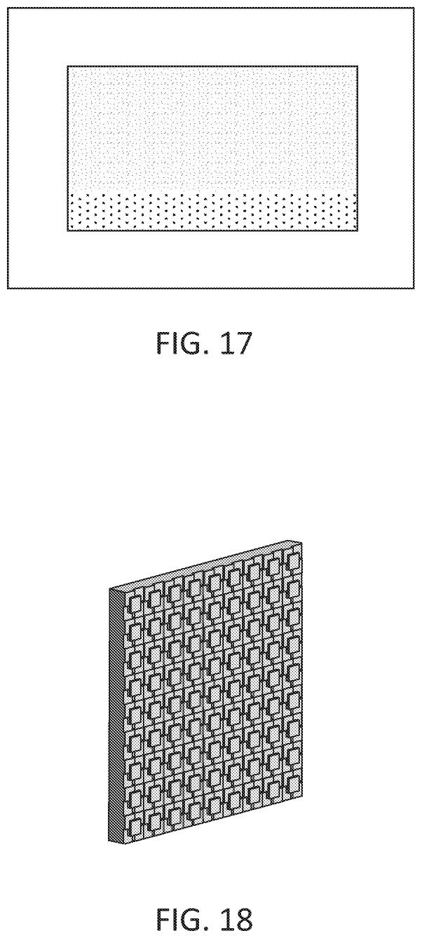

FIG. 17 is a partial front elevation view of an operating acoustic separation device showing an acoustic edge effect. The view is taken through a transparent reflector.

FIG. 18 is an isometric view of a faceted reflector. The faceted reflector reflects acoustic waves from the transducer to produce acoustic standing waves in a fluid chamber or flow path. The faceted reflector can contribute to increasing the gradients in the acoustic field generated by the acoustic standing wave. Flat or faceted reflectors can be used to establish the acoustic edge effect. Faceted reflectors can maintain multiple parallel standing waves, increase diffraction of waves, which increases gradients in the acoustic field thereby increasing radiation forces, particularly in the lateral direction. The pressure profiles from simulations show scattered pattern of the wave field that changes with frequency. The pressure scattering give rise to larger gradients in pressure, leading to higher radiation forces, especially in the lateral directions.

In an example experiment, a column is loaded with 15% of 5 um droplets and the edge effect is established. Jurkat cells are flowed in and the flush fractions are measured to determine their wash out. There is no binding in this experiment. At the same low power to flow rate ratio, the faceted reflector shows more efficient washout of cells compared to a flat reflector. With the faceted reflector, cells are trapped in the acoustic field, whereas for the same power using a flat reflector, there is no visible evidence of trapped cells in the acoustic field, indicating cells are flushed through the acoustic field more efficiently. It takes 5 column volumes (CVs) to get to the asymptotic value of cell flush-out for the configuration with the faceted reflector, and at least 13 CVs to reach the same flush-outs for the flat reflector. Faceted reflectors show an ability to work at lower power to flow rate ratio than the flat reflector.

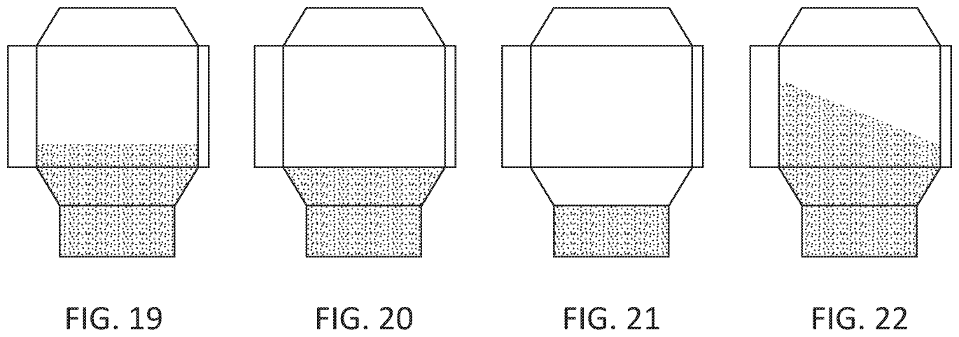

FIGS. 19, 20, 21, and 22 are partial front elevation views of an operating acoustic separation device showing different acoustic interface region locations obtained with different example experiments. The experiments were run with a flat reflector and a 1.5″×1.5″ transducer. At lower power, the interface forms at or slightly above the acoustic edge, as shown in FIG. 19. As the power is increased, the interface is pushed down and forms below the acoustic field as shown in FIG. 20. The distance from the acoustic edge where the interface forms increases as the power is increased, as further illustrated in FIG. 21. At a lower power to flow rate ratio and higher flow rate, the interface forms inside the acoustic field rising up near the middle section of the acoustics, as illustrated in FIG. 22. In FIGS. 19-22, the transducer is on the left side and the reflector is on the right side. The interface may form at an angle from transducer to reflector as clearly seen in FIG. 22.

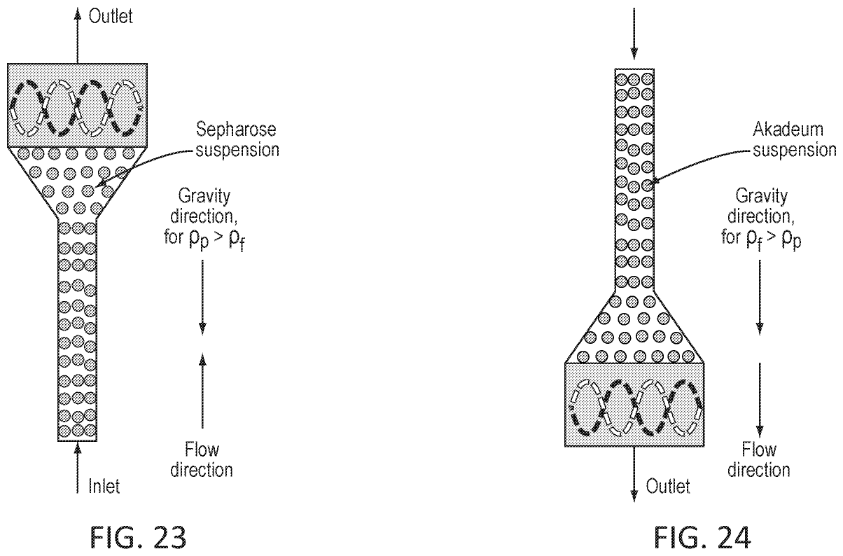

FIG. 23 is a diagram of an acoustic separation device implementing an acoustic edge effect for particles with a density greater than water. FIG. 24 is a diagram of an acoustic separation device implementing an acoustic edge effect for particles with a density less than water. A number of example experiments were run using the configurations of FIG. 23 and FIG. 24, The column geometry is of importance for the acoustofluidic behavior of the column. A diffuser at a top of the column allows easier settling of particles helping the “self clearing” mechanism so that the acoustic edge does not saturate. Parameters like diffuser height, diffuser angle, column diameter play a role in column operation. The column can also be run with a straight section without a diffuser. In the interface region there is a net zero balance of particle flow; as new particles are flowed into the interface region by the flow field, an equal and opposite flux of particles exist. Multiple mechanisms exist for this opposite particle flux. For example, the diffuser at the top of the fluidized column allows for recirculating flow containing a downward flow. Acoustic streaming may set up counterrotating vortices that contain downward flow. Additional angled surfaces within the fluidized bed create enhanced gravitational settling as particles can slide down these surfaces.

The particles can be solid, droplets or bubbles, for example. If the particles are heavier or denser than the fluid, the system is operated in a vertically upwards configuration. If the particles are lighter or less dense than the fluid, the system is operated in a vertically downwards configuration. In both scenarios, the gravity force on the particles helps formation of the interface region. Examples of solid particles include microcarriers, PROMEGA beads, and polymer particles. Examples of liquid particles include PFH droplets and PFP droplets. Examples of bubbles include microbubbles, Akadeum beads, hollow glass beads. As used herein, particles refers to all types of materials used to form beads, including solids, liquids, hollow materials, materials denser than water and less dense than water, and any type of material that can be formed into discrete portions that can be entrained in a fluid flow.

The behavior of the acoustic edge is different for particles with different properties. A four quadrant classification is used to identify the different properties and acoustic edge effect behavior. In a first quadrant, X>0, ρₚ>ρ_{f} representing beads with a density greater than water and a positive acoustic contrast factor. These beads include Promega beads and microcarriers. A second quadrant includes particles with a properties of X>0, ρ_{f}>ρₚ where the beads have a negative contrast factor and are less dense than water. A third quadrant includes particles with the properties of X<0, ρₚ>μ_{f} where the beads have a negative acoustic contrast factor and are denser than water. These beads include PFH droplets. A fourth quadrant has particles with the properties of X<0, ρ_{f}>ρₚ where the beads are less dense than water and have a positive acoustic contrast factor. These beads include Akadeum beads and hollow glass spheres.

In each of the cases in all four quadrants, an acoustic edge interface can be formed. The flow direction is opposite to the direction of the gravity/buoyancy force. The location of the acoustic edge may be different for different contrast factor particles.

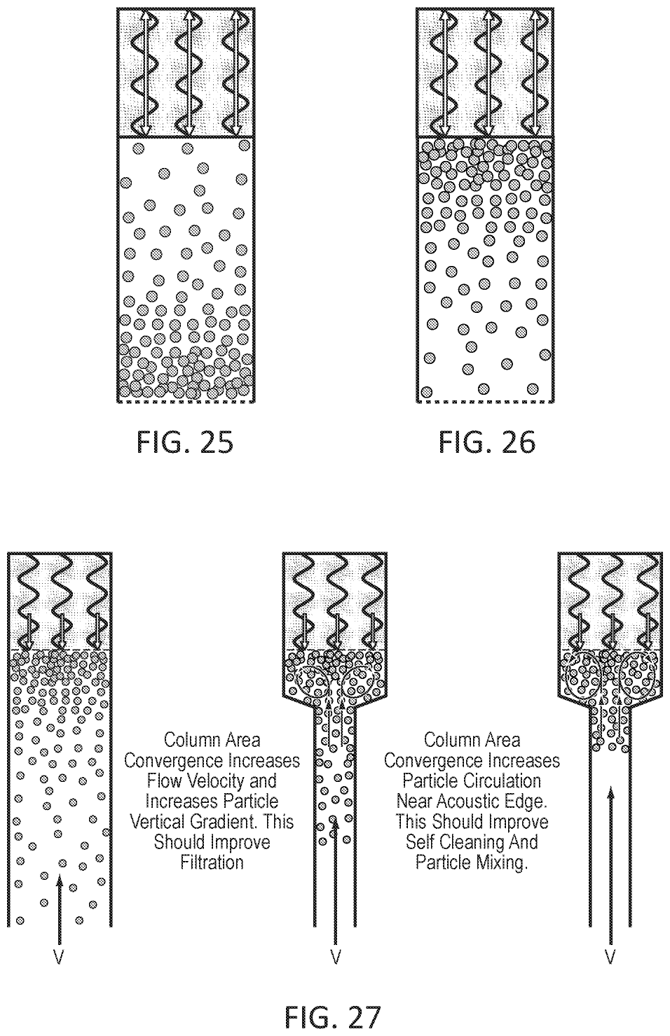

FIG. 25 is a diagram of an acoustic separation device implementing an acoustic edge effect with no fluid flow. FIG. 26 is a diagram of an acoustic separation device implementing an acoustic edge effect with fluid flowing. A multi-directional acoustic field is implemented at an end of the column. The lateral standing wave force field and wave propagation for a given frequency operation of the transducer are illustrated. A fixed number of particles in suspension in a column of fluid is shown. Particles can be solid, liquid or gaseous. Particles here are assumed to be heavier than the fluid. Gravity is acting down vertically. The acoustic edge effect is established as follows. As flow is initiated upward through the particle mixture, acoustic radiation forces form an interface between mixture of particles and fluid on one side and clear fluid on the other side. The radiation forces in the flow direction arise from the lateral standing wave field component of the multidirectional field. Acoustic radiation forces exert a downward force on the particles which is stronger than the fluid drag forces on the particles, potentially aided by the gravitational force, thereby maintaining a steady interface. The acoustic edge can be formed at the lower edge of the acoustic standing wave, and can be in the acoustic field as well. As shown in FIG. 26, the dense particle concentrations near the acoustic edge is used to filter any flowing fluid, or flowing fluid mixture.

High column particle density near acoustic edge acts as a filter. Particle size, density with system acoustic power and flow rate can be varied to generate extreme filtration. The unsteady acoustic wave reflection generates particle movement and self cleaning. The filtering particles in the column can be varied using contrast factor, size or density factor. Smaller column particles can filter smaller target particles and multiple size column particles can further intensify the filtering. The column may have a concentration gradient in the flow direction depending on the properties of the particles but is not necessary for the operation of the acoustic edge. Multiple size column particles, with proper contrast factor, and density can be used to conduct extreme filtering. There are multiple control variables to filter almost any size particle. Such extreme filtering can potentially be used for filtering exosomes and viruses. For example, smaller particles can be chosen to have a lower acoustic contrast factor so that they are maintained at the interface region more readily than larger particles or particles with a greater acoustic contrast factor. By maintaining smaller particles at the interface region, the interstices between the particles can be reduced to provide for very small pore extreme physical filtering.

The location of the interface region can be modified in accordance with various influential parameters, as discussed above. For example, and angled interface region can be implemented, similar to that illustrated in FIG. 29. In such implementations, the lateral radiation force can be enhanced since a component of primary force acts perpendicular to the interface. The lateral force on the interface is seen to be negative for most of the frequencies, meaning that the interface will be held back and particles will be stopped from moving in the flow direction (which is in the positive direction).

FIG. 27 is a diagram of acoustic separation devices with varying geometries and fluid dynamics. Flow velocity at acoustic edge is held constant in all three configurations. Column area convergence increases flow velocity and increases particle vertical gradient. Such convergence can improve filtration. As column area convergence increases, particle circulation occurs near the acoustic edge. The particle circulation can improve “self-cleaning” and aid with particle mixing.

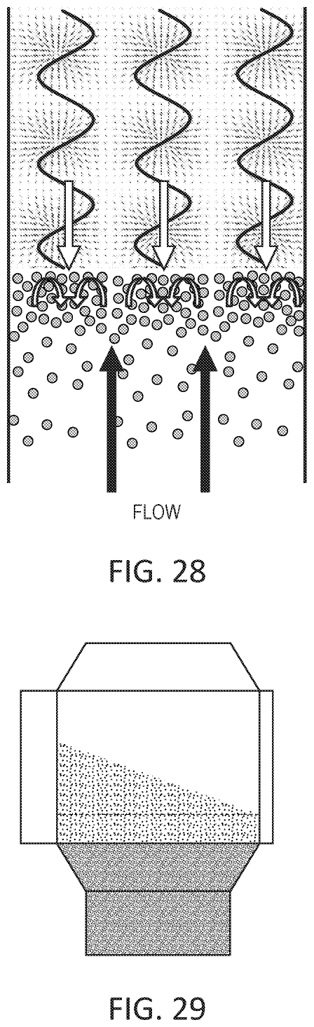

FIG. 28 is a diagram of an acoustic separation device illustrating vortices at an acoustic interface region. Stirring vortices set up by lateral waves reflecting off the particle interface and/or acoustic radiation force variations on the particles results in a self-cleaning acoustic filter interface. Acoustic streaming is a steady flow in a fluid driven by Reynolds stresses and the absorption of acoustic waves. High power can cause wave attenuation when moving through a fluid where the sound wave is absorbed in the medium of propagation. This results in a body force on the fluid driving the fluid motion inside the cavity. The more a wave attenuates the stronger the streaming. If the particles are small enough, they will move with the bulk streaming flow. This behavior has been observed in the unit especially when the particle mixture rises up in the acoustic field with a smaller pathlength. Acoustic streaming could also help in generate the acoustic edge by the action of counter rotating vortices that keep the interface from rising up. Acoustic streaming is stronger in a traveling wave setup and edge is unstable when the particles rise up in the acoustic field. Acoustic streaming may also provide additional mixing and a self clearing mechanism for the particles as long as it is stable and controllable.

FIG. 29 is a partial front elevation view of an acoustic separation device with multiple interface regions. It is possible to establish multiple edge effects in the same system. FIG. 29 demonstrates an edge effect on a mixture of 6% VF leukopak and 15% VF droplets. Since the droplets have a stronger acoustic response, the edge is set up at the bottom of the acoustic field and all droplets are held below it. The cells like RBCs have weaker acoustic response compared to the droplets and they rise up in the acoustic field. Since a transducer typically provides higher pressures in the center, it is possible to establish an edge with cells at halfway into the acoustic field. This way multiple edge effects can be created with in the same system, for particles of different properties. This can be a way of separate particles with different acoustic responses using the edge effect and has been shown to deplete platelets in a leukopak by 70% with a 70% recovery of the WBCs.

It is possible to establish edge effect for only one particle type. This way the particles with lower acoustic response can be flowed through or flushed out from the column. As the concentration of particles that form the acoustic edge is increased, the particles may start to act like a hydrodynamic filter. This way larger particles can be retained and smaller particles like viruses or platelets could be passed through.

Experiments were conducted to perform separation on undiluted leukopaks (5 ml volume) run in the fluidized bed without any affinity particles. Different power and flow rate conditions were run in the experiments. The results show that the edge was established for RBCs and WBCs. Platelets being smaller (3 um), pass through to the outlet. Six to eight column flushes were performed and 80% recovery of WBCs with 68% depletion of platelets was observed. Some acoustic retention of platelets was seen. The numbers can be tweaked by tuning power and flow rates to get desired recovery and/or depletion. Similar idea can be used for other particles like exosomes, viruses, cell debris etc.

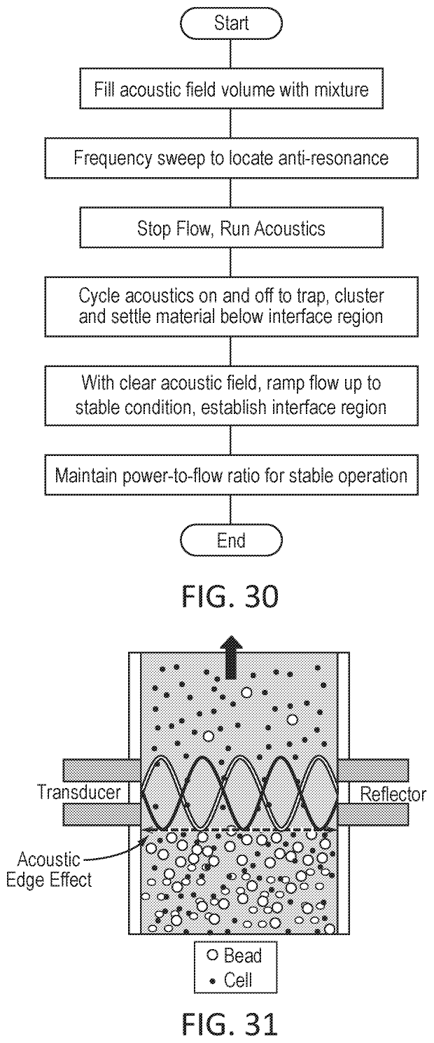

FIG. 30 is a flowchart of a process for initiating an acoustic edge effect. Initially the acoustic field is filled with particle mixture. A frequency sweep is performed and the antiresonance (point close to zero reactance or maximum resistance is selected to run the experiment. The flow is stopped and acoustics are turned on, and operated in a multidimensional standing wave field. By periodically turning the acoustics on and off, the particles in the acoustic field are trapped, clustered and settled below the acoustic edge. Once most of the particles are out of the acoustic field, the flow is slowly turned on and ramped up to a stable condition. Once the interface is established, and as long as a proper power to flow rate ratio is maintained, the system can run in a stable manner.

FIG. 31 is a diagram of an acoustic separation device configured for affinity separation of cellular material. The beads are held back by the acoustic field generated at the interface region.

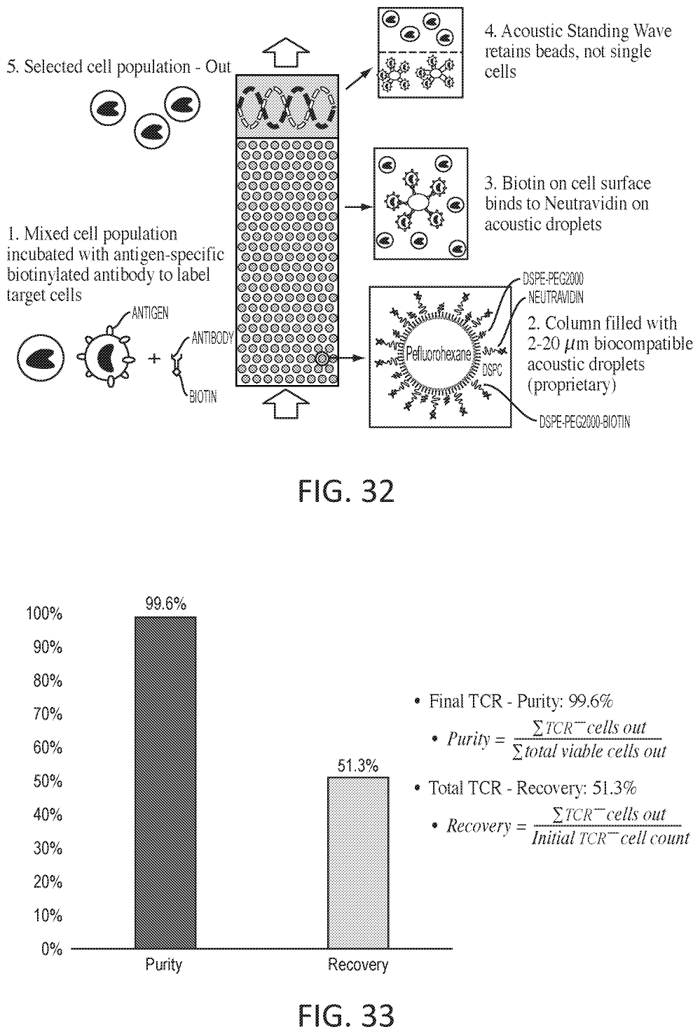

FIG. 32 is a diagram of an acoustic separation device and process for affinity separation of cellular material with perfluorohexane droplets.

FIG. 33 is a graph of purity and recovery for TCR—cellular material. TCR-depletion can be implemented using the acoustic separation techniques described herein. In one experiment, the system is loaded with 15% functionalized droplets by volume, TCR antibody is used to capture CD3+ cells. Diluted and undiluted leukopaks are flowed in and the flush fractions are analyzed for different cells types where non-target WBCs are CD3−. The system is very efficient in flushing out RBCs and platelets (>90% flushout). The system shows some retention of non-target WBCs like monocytes, granulocytes, B cells or NK cells. Some of these cells such as monocytes are bigger than target T-cells and may be trapped acoustically. Some bigger cells may also be trapped within the fluidized bed. With dilution, higher percentage of non-target cells are flushed out. This can be because of wider fluidic channels available because of diluted feed. The experimental data demonstrates that the acoustic fluidized bed can retain the particles/cells of interest, but flush out particles/cell not of interest, as long as there is sufficient acoustic differentiation between the different constituents.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known processes, structures, and techniques have been shown without unnecessary detail to avoid obscuring the configurations. This description provides example configurations only, and does not limit the scope, applicability, or configurations of the claims. Rather, the preceding description of the configurations provides a description for implementing described techniques. Various changes may be made in the function and arrangement of elements without departing from the spirit or scope of the disclosure.

Also, configurations may be described as a process that is depicted as a flow diagram or block diagram. Although each may describe the operations as a sequential process, many of the operations can be performed in parallel or concurrently. In addition, the order of the operations may be rearranged. A process may have additional stages or functions not included in the figure.

Having described several example configurations, various modifications, alternative constructions, and equivalents may be used without departing from the scope of the disclosure. For example, the above elements may be components of a larger system, wherein other structures or processes may take precedence over or otherwise modify the application of the invention. Also, a number of operations may be undertaken before, during, or after the above elements are considered. Accordingly, the above description does not bound the scope of the claims.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

1. A process for separating materials from a fluid mixture, the process comprising:
flowing a fluid mixture containing the materials into a chamber;
generating an acoustic wave with an acoustic transducer at one end of the chamber;
establishing an interface region near the acoustic wave; and
blocking the materials from flowing with the fluid using an acoustic field generated at the interface region.

2. The process of claim 1, wherein the materials are trapped by a multi-directional acoustic standing wave.

3. The process of claim 1, wherein a pressure rise and an acoustic radiation force on the materials are generated at the interface region.

4. The process of claim 1, further comprising recirculating fluid passing through the acoustic wave back to the chamber.

5. The process of claim 1, wherein the multi-directional acoustic standing wave results in an acoustic radiation force having an axial force component and a lateral force component that are of the same order of magnitude.

6. An acoustic separation device, comprising:
an ultrasonic transducer coupled to an end of a chamber suitable for containing a fluid flow;
a controller coupled to the ultrasonic transducer and configured to cause the ultrasonic transducer to generate an acoustic wave at the end of the chamber, such that the acoustic wave has an acoustic field with gradients in a number of directions;
a fluid pump configured to generate a fluid flow in the chamber;
material in the fluid flow interacting with the gradients to form an interface region that blocks the material from passing through the acoustic field.

7. The device of claim 6, further comprising a reflector to reflect the acoustic wave to generate an acoustic standing wave.

8. The device of claim 7, wherein the reflector further comprises facets.

9. The device of claim 7, wherein the acoustic standing wave is a multi-directional acoustic standing wave.

10. The device of claim 6, wherein the acoustic transducer is configured to be excited in a higher order mode.

11. The process of claim 1, wherein the chamber is a vertically oriented column and further comprising utilizing gravity or buoyancy forces to contribute to forming the interface region.

12. The process of claim 1, wherein establishing the interface region further comprises reducing lateral wave force near an upstream side of the acoustic wave.

13. The process of claim 1, further comprising increasing a concentration of the material on the upstream side of the acoustic wave.

14. The process of claim 13, wherein an increased concentration of the material on the upstream side of the acoustic wave forms a pressure differential that contributes to forming the interface region.

15. The process of claim 1, further comprising forming fluids with different characteristics on either side of the interface region.

16. The device of claim 6, further comprising a tunable barrier at the interface region.

17. The device of claim 6, wherein the controller is configured to control the acoustic wave to control characteristics of the interface region.

18. The device of claim 6, wherein the chamber further comprises a vertically oriented column.

19. A method for separating material from a fluid mixture, comprising:
flowing a fluid mixture containing the material into a chamber in a vertical direction;
actuating an acoustic transducer located at one end of the chamber to generate an acoustic wave across the chamber; and
forming an interface region on an upstream side of the acoustic wave, such that a material concentration is greater upstream of the acoustic wave.

20. The method of claim 19, further comprising forming a pressure differential at the interface region.
